(19) United States
(12) Patent Application Publication
(10) Pub. No.: US 2009/0275800 A1
(43) Pub. Date: Nov. 5, 2009

(54) CAMERA FOR POSITIONING IN THE PYLORUS DURING BARIATRIC PROCEDURES

(76) Inventor: Mark S. Zeiner, Mason, OH (US)

Correspondence Address: WELSH & FLAXMAN LLC, 2000 DUKE STREET, SUITE 100, ALEXANDRIA, VA, 22314, US

(21) Appl. No.: 12/113,699

(22) Filed: May 1, 2008

(51) Int. Cl. A61B 1/273

(52) U.S. Cl. 600/115

(57) ABSTRACT

A lumenal camera assembly includes housing and a pill cam supports the housing. The housing is shaped and dimensioned for placement within the pylorus.

### BACKGROUND OF THE INVENTION

1. Field of the Invention

The invention relates to bariatric surgery. More particularly, the invention relates to a visualization device that may be incorporated into the gastric system for visualizing the stomach during gastric procedures.

2. Description of the Related Art

Obesity is a medical condition affecting more than 30% of the population in the United States. Obesity affects an individual's personal quality of life and contributes significantly to morbidity and mortality. Obese patients, i.e., individuals having a body mass index (“BMI”) greater than 30, often have a high risk of associated health problems (e.g., diabetes, hypertension and respiratory insufficiency), including early death. With this in mind, and as those skilled in the art will certainly appreciate, the monetary and physical costs associated with obesity are substantial. In fact, it is estimated the costs relating to obesity are in excess of 100 billion dollars in the United States alone. Studies have shown that conservative treatment with diet and exercise alone may be ineffective for reducing excess body weight in many patients. Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In an RYGB procedure a small stomach pouch is separated from the remainder of the gastric cavity and attached to a resectioned portion of the small intestine. This resectioned portion of the small intestine is connected between the “smaller” gastric cavity and a distal section of small intestine allowing the passage of food therebetween. The conventional RYGB procedure requires a great deal of operative time. Because of the degree of invasiveness, post-operative recovery can be quite lengthy and painful. Still more than 100,000 RYGB procedures are performed annually in the United States alone, costing significant health care dollars.

Morbid obesity is defined as being greater than 100 pounds over one's ideal body weight. For individuals in this category, RYGB, gastric banding or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing a feeling of satiety. A band is placed around the stomach near the junction of the stomach and esophagus. The small upper stomach pouch is filled quickly, and slowly empties through the narrow outlet to produce the feeling of satiety. Other forms of bariatric surgery that have been developed to treat obesity include Fobi pouch, bilio-pancreatic diversion and gastroplasty or “stomach stapling”.

It is also known to create cavity wall plications through endoscopic only procedures. However, operating solely within the interior of the gastric cavity limits the plication depth that can be achieved without cutting. Furthermore, access and visibility within the gastric and peritoneal cavities is limited in a purely endoscopic procedure as the extent of the reduction increases.

With the foregoing in mind, it is desirable to provide surgical weight loss procedures (and related medical instruments) that are inexpensive, with few potential complications, and that provide patients with a weight loss benefit while buying time for the lifestyle changes necessary to maintain the weight loss. Further, it is desirable that the procedures be minimally invasive to the patient, allowing for a quick recovery and less scarring. The present invention provides a medical instrument effective in improving the performance of gastric procedures.

### SUMMARY OF THE INVENTION

It is, therefore, an object of the present invention to provide a lumenal camera assembly including a housing and a pill cam supported by the housing. The housing is shaped and dimensioned for placement within the pylorus.

It is also an object of the present invention to provide a lumenal camera assembly including a control tube secured to a back end of the lumenal camera assembly.

It is another object of the present invention to provide a lumenal camera assembly wherein the housing includes a first end and a second end. The first end is provided with a first balloon member and the second end is similarly provided with a second balloon member.

It is a further object of the present invention to provide a lumenal camera assembly wherein the first balloon member and the second balloon member are substantially annular and are shaped and dimensioned for selective engagement with body members through which the lumenal camera assembly passes.

It is another object of the present invention to provide a lumenal camera assembly including a control system for inflation and deflation of the first balloon member and the second balloon member.

It is a further object of the present invention to provide a lumenal camera assembly wherein the housing includes a housing body which extends from the first balloon member to the second balloon member, and a third balloon member is mounted about the housing body for movement between the first balloon member and the second balloon member.

It is also an object of the present invention to provide a lumenal camera assembly including a control system for inflation and deflation of the first balloon member, the second balloon member and the third balloon member.

It is another object of the present invention to provide a lumenal camera assembly wherein the first balloon member, the second balloon member, and the third balloon member are substantially annular.

It is a further object of the present invention to provide a lumenal camera assembly wherein the pill cam is mounted within the housing for movement between the first end of the housing and the second end of the housing.

It is also an object of the present invention to provide a lumenal camera assembly wherein the pill cam is secured to the third balloon member such that the third balloon member is moved longitudinally along the housing with the pill cam.

It is also an object of the present invention to provide a lumenal camera assembly including a mechanism for insufflation or suction.

It is another object of the present invention to provide a method for visualization of the gastric cavity. The method includes transorally deploying a lumenal camera assembly composed of a housing with a pill cam mounted thereto and positioning the lumenal camera assembly within the pylorus such that the pill cam is capable of visualizing the gastric cavity.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic view showing the present lumenal camera assembly positioned within the pylorus region of a patient.

FIG. 2 is a perspective view of the lumenal camera assembly with the pill cam adjacent the front end of the housing.

FIG. 3 is a perspective view of the lumenal camera assembly with the pill cam positioned at the back end of the housing.

FIGS. 4 and 5 are respectively side views showing the lumenal camera assembly as shown in FIGS. 2 and 3, respectively.

FIG. 6 is a cross sectional view along the line 6-6 in FIG. 5.

FIGS. 7, 8, 9 and 10 show the various steps associated with placement of the lumenal camera assembly within the pylorus of the stomach.

FIGS. 11 through 21 show manipulation of the lumenal camera assembly within a vessel.

FIG. 22 is a cross sectional view of the lumenal camera assembly.

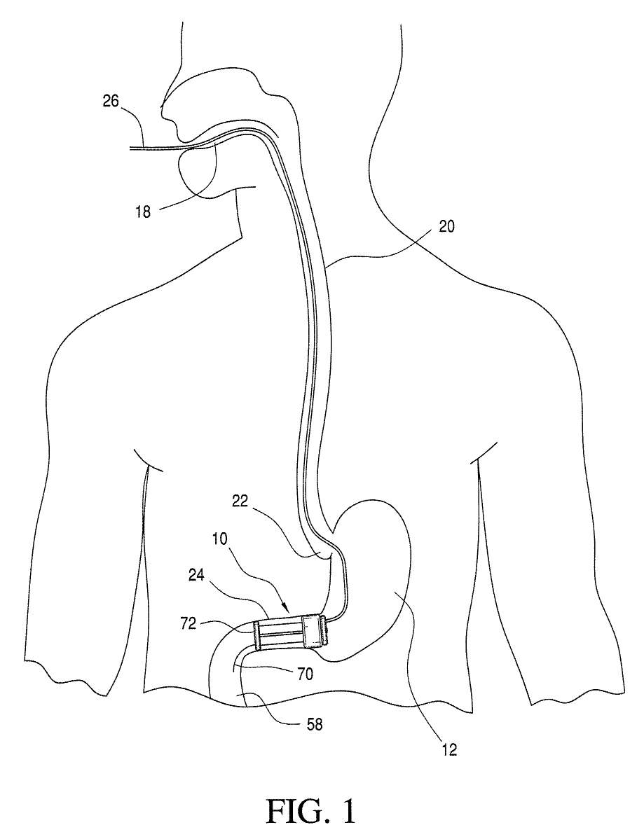

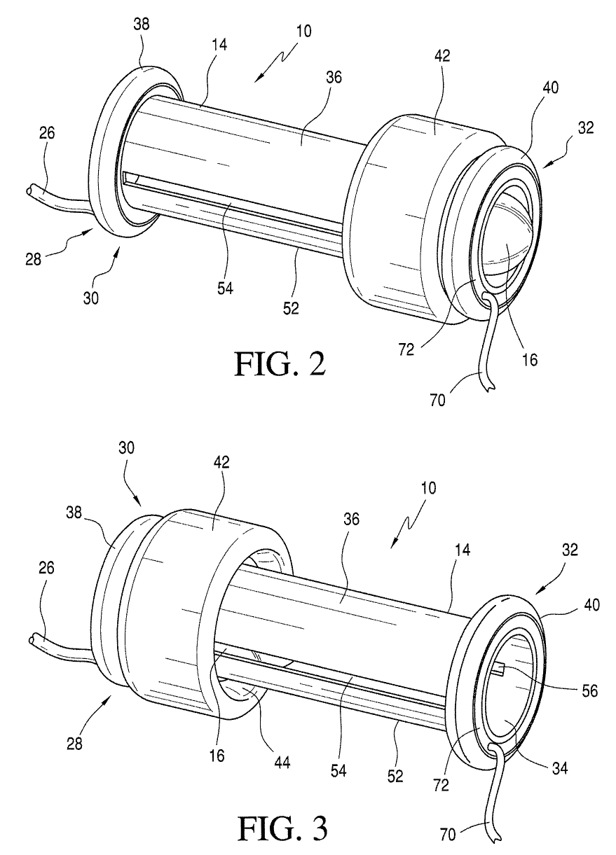

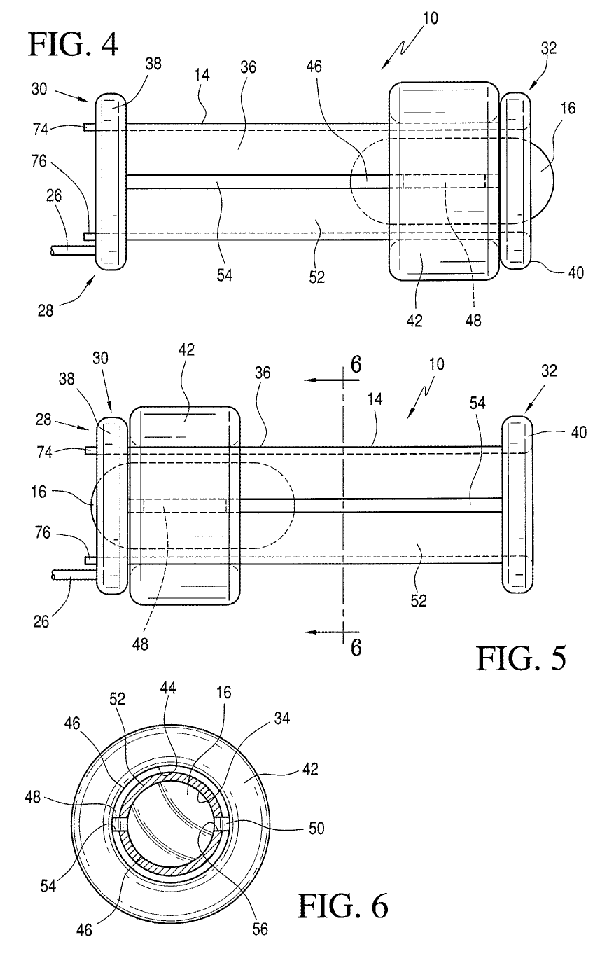

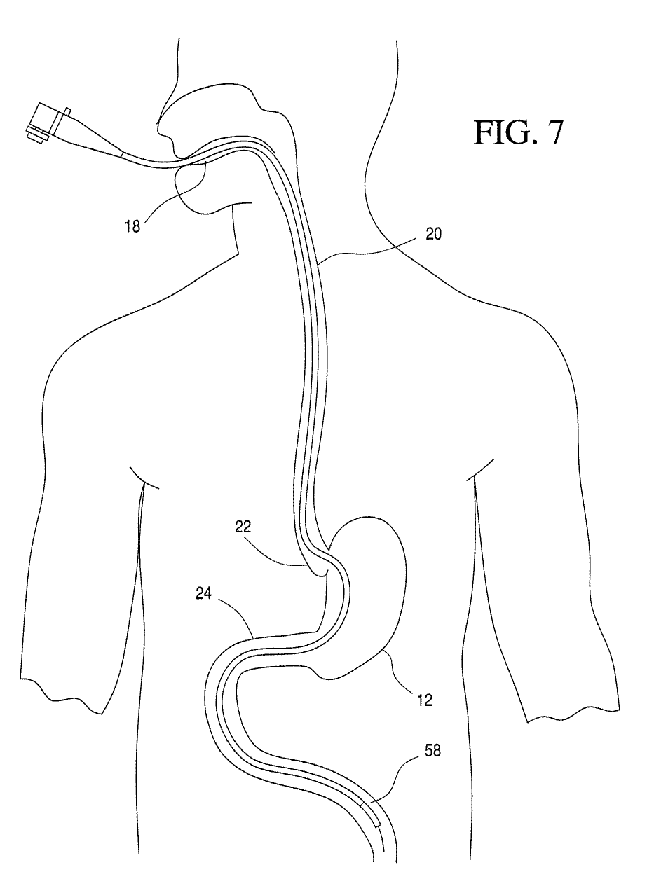

### DESCRIPTION OF THE PREFERRED EMBODIMENT

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as a basis for teaching one skilled in the art how to make and/or use the invention.

With reference to the various figures, a lumenal camera assembly 10 for use in bariatric gastroplasty restriction procedures is disclosed. The lumenal camera assembly 10 provides for visualization that can be utilized to view the internal structure of the stomach 12 while the gastric volume reduction procedure is taking place while also draining stomach liquids during a bariatric gastroplasty. As the following disclosure also makes clear, the present lumenal camera assembly 10 functions as a plug to prevent fluid transfer through the pylorus and into the small bowel. Although the present lumenal camera assembly 10 is described herein in accordance with a preferred embodiment thereof for use in the performance of gastric volume reduction procedures, it is contemplated the present lumenal camera assembly 10 may be used in a variety of surgical procedures without departing from the spirit of the present invention.

Briefly, the lumenal camera assembly 10 includes a housing 14 which supports a pill cam 16, that is, a camera in the shape and form of a pill (capsule) such that it is adapted for introduction within the body via the mouth in the same manner a patient would take a conventional pill. The lumenal camera assembly 10 is inserted into the mouth 18 and guided down into the esophagus 20 using an alternating balloon inflation process described below in greater detail. The lumenal camera assembly 10 continues through the esophagastric junction 22 and into the stomach 12. Once the lumenal camera assembly 10 is positioned within the stomach 12, it is manipulated into the pyloric canal 24 and is placed with one end lodged into the pylorus 24 with the lumenal camera assembly 10 looking back into the stomach 12 for viewing of the internal structure of the stomach 12 during the gastric volume reduction procedure. The diameter of the lumenal camera assembly 10 may also be small enough to make it possible to insert it through the esophagus without using the alternating balloon inflation process described below. It is further contemplated the alternating balloon inflation process may be employed to advance the lumenal camera assembly 10 into the pylorus.

As will be discussed below in greater detail, and in accordance with a preferred embodiment of the present invention, a multi-lumen control tube 26 is inserted into the back end 28 of the lumenal camera assembly 10 to assist in its introduction into the stomach 12. With the control tube 26 secured to the back end 28 of the lumenal camera assembly 10, the lumenal camera assembly 10 may be utilized as an endoscope. It is also contemplated an endoscope may be employed in deployment of the lumenal camera assembly 10. Although the control tube 26 is fixedly coupled to the lumenal camera assembly 10 in accordance with a preferred embodiment of the present invention, it is contemplated once the lumenal camera assembly could be designed such that once it is properly positioned within the pylorus with the pill cam looking back into the stomach, the control tube may be removed for permanent placement of the lumenal camera assembly 10 in the gastric system during the procedure. In accordance with such an alternate embodiment, the control tube would be simply pushed into the back end of the lumenal camera assembly creating a loose slip fit. Since there would be no actual connection means other than a loose slip fit, removal and disconnection of the control tube would be easy, as all one needs to do is pull on the control tube 26 and it will release from the back end of the lumenal camera assembly 10. In addition, it is contemplated a flexible endoscope can be used in place of the control tube.

In accordance with a preferred embodiment, the lumenal camera assembly 10 includes a housing 14 having a first (or back) end 30 and a second (or front) end 32. The housing 14 includes an internal surface 34 shaped and dimensioned for supporting the pill cam 16 for longitudinal movement within the housing 14 and between the first end 30 and second end 32 thereof. The housing 14 also includes an outer surface 36 provided with various mechanisms used in the placement and operation of the present lumenal camera assembly 10.

The first end 30 is provided with a first balloon member 38 mounted along the outer surface 36 of the housing 14 and the second end 32 is similarly provided with a second balloon member 40 mounted along the outer surface 36 of the housing 14. Both the first balloon member 38 and the second balloon member 40 are substantially annular and are shaped and dimensioned for selective engagement with body members through which the present luminal camera assembly 10 passes. The first and second balloon members 38, 40 are selectively inflated and deflated to allow for controlled positioning of the lumenal camera assembly 10 in a desired manner as described herein. The housing 14 further includes a housing body 52 that extends from the first balloon member 38 to the second balloon member 40. A third balloon member 42 is mounted about the housing body 52 for movement between the first balloon member 38 and the second balloon member 40. As with the first balloon member 38 and the second balloon member 40, the third balloon member 42 is substantially annular and is shaped and dimensioned for selective engagement with body members through which the present lumenal camera assembly 10 passes.

A control system 60 composed of a control computer 62, a vacuum pump 64 and an inflation pump 66 is connected to the lumenal camera assembly 10 via the multi-lumen control tube 26 to control inflation/deflation of the first, second and third balloon members 38, 40, 42. As those skilled in the art will appreciate, a fluid and/or mechanical and/or electrical system may be used to systematically inflate and deflate the balloon members 38, 40, 42 which advances or retracts the lumenal camera assembly 10 automatically to position the pill cam 16 so that it can be used to view desired internal anatomical positions. As discussed above, a multi-lumen control tube 26 connects the control system 60 to the lumenal camera assembly 10 to deliver all the necessary pressures and control mechanisms to the lumenal camera assembly 10. The control system 60 includes a control computer 62, at least two pressure generation pumps 64, 66, mechanical force generating capability, either rotational or linear motion, and multiple valves that deliver differing fluid pressures to the lumenal camera assembly 10 through the multi-lumen control tube 26.

The pill cam 16 is mounted within the housing 14 for movement between the first end 30 of the housing 14 and the second end 32 of the housing 14. The pill cam 16 is a commercially available device and the preferred device comes from Given Imaging Ltd. It is contemplated the pill cam could be replaced with a camera that is connected to the control unit to provide a live video image rather than a series of wireless transmitted photos. As such, it should be understood that while pill cam technology is employed in accordance with a preferred embodiment of the present invention, live video technology may be interchangeably utilized without departing from the spirit of the present invention.

The pill cam 16 is secured to the third balloon member 42 in a manner allowing the pill cam and third balloon member 42 to move between the first end 30 and the second end 32 of the housing 14. In particular, the third balloon member 42 includes an inner surface 44 and the pill cam 16 includes an outer surface 46. First and second support members 48, 50 extend between and couple together the inner surface 44 of the third balloon member 42 and the outer surface 46 of the pill cam 16. The first and second support members 48, 50 are diametrically opposed with regard to their positioning along the outer surface 46 of the pill cam 16. With this in mind, the housing body 52 includes first and second slots 54, 56 through which the respective first and second support members 48, 50 extend for connecting the third balloon member 42 to the pill cam 16. Since the third balloon member 42 may be moved longitudinally along the housing 14 with the pill cam 16 secured thereto, the pill cam 16 is capable of being moved between a storage position at the second (that is, the front) end 32 of the housing 14 in which the pill cam 16 is stored within the housing 14 and an exposed position at the first (that is, the back) end 30 of the housing 14 in which it may be utilized for visualization of the stomach 12.

In accordance with a preferred embodiment, energy is transferred from the control system 60 outside the body traveling down the multi-lumen control tube 26 to the lumenal camera assembly 10. Movement of the pill cam 16 within the housing 14 is controlled by a linear motion device 80 for example, a roller driven mechanism causing relative motion between the pill cam 16 and the housing 14 by employing a screw driven by a servomotor to produce linear motion where a servomotor thereof is controlled from the control system 60. In all of these configurations linear motion (as controlled by signals transmitted through the multi lumen control tube 26) is used to move the support members 48, 50 within the first and second slots 54, 56 (and ultimately move the third balloon member 42 and the pill cam 16). Further, and in accordance with an alternate embodiment, it is contemplated a light duty spring may be used to bias the pill cam 16 proximally so the control tube or flexible endoscope is used to push on the spring loaded pill cam device for delivery into the pylorus.

As briefly mentioned above, the present lumenal camera assembly 10 may be utilized as a blockage device for blocking and obstructing the small bowel 58 from the stomach 12 during surgical procedures. In addition, the lumenal camera assembly 10 may also be fitted with a suction drain to keep fluids from entering the intestinal track. In accordance with a preferred embodiment, the multi lumen control tube 26 is connected to a suction tube 70 extending from the distal tip 72 of the lumenal camera assembly 10 at the second end 32 of the housing 14. As such, the suction tube 70 would be in fluid communication with the operating room suction system for controlled suction of fluids that enter the intestinal track. After the lumenal camera assembly 10 is successfully placed inside the pylorus 24, the suction is turned on at a very low pressure so that the small bowel is continuously purged of any fluids that escape beyond the plugged pylorus 24. In addition, the lumenal camera assembly 10 is also provided with an insufflation tube 84 and a suction tube 86 mounted along the first end 30 of the housing 14, and in fluid communication with the multi-lumen control tube 26 for respective attachment to a pressure source and a suction source.

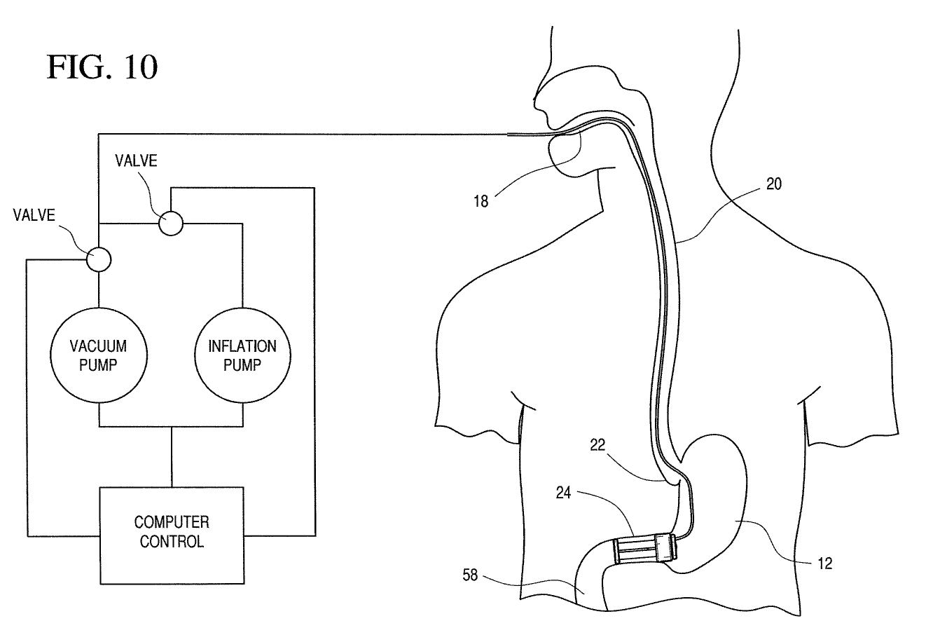

As discussed above, the lumenal camera assembly 10 of the present invention is ultimately positioned within the pylorus 24 for visualization of the gastric cavity. Referring to FIGS. 7, 8, 9 and 10, a sequence for deployment of the present lumenal camera assembly 10 is disclosed. First, and with reference to FIG. 7, a flexible endoscope 74 is inserted deep within the gastrointestinal tract beyond the duodenum and a flexible guide wire 76 is then inserted deep into the gastrointestinal tract. The endoscope 74 is then removed.

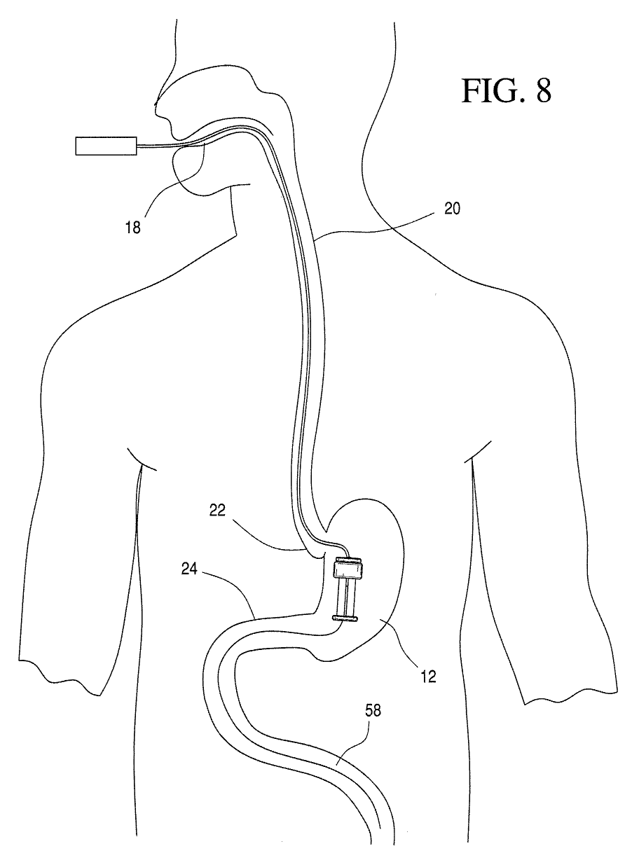

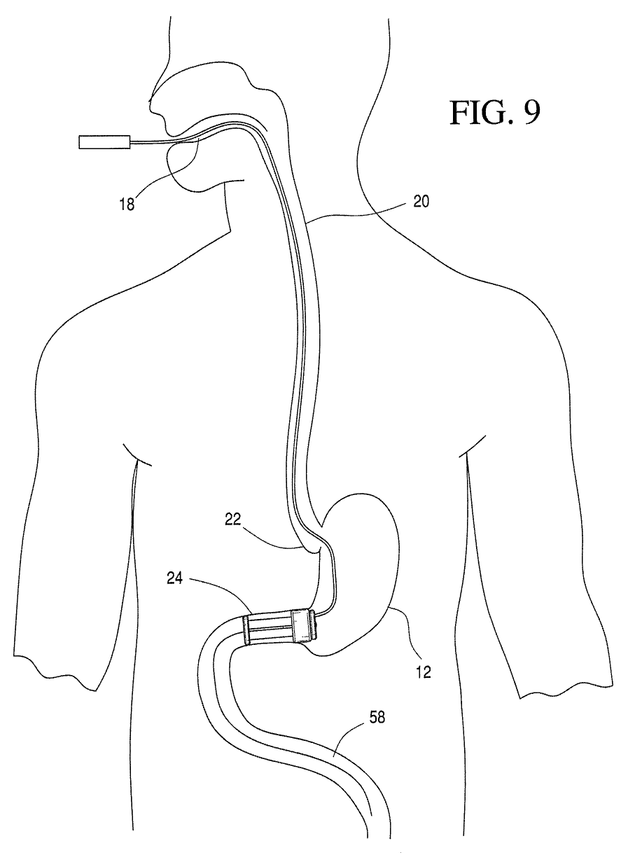

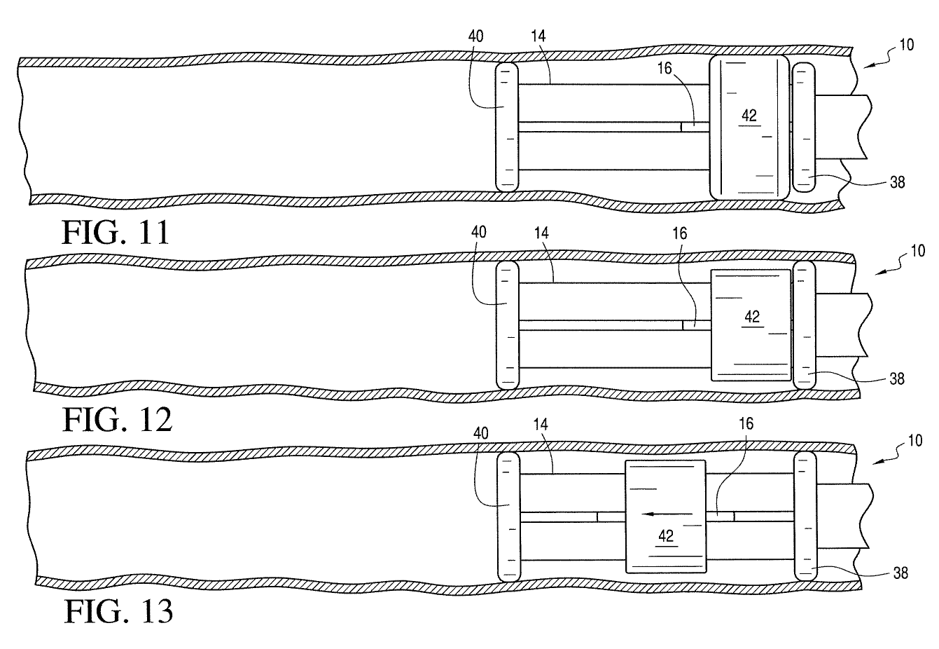

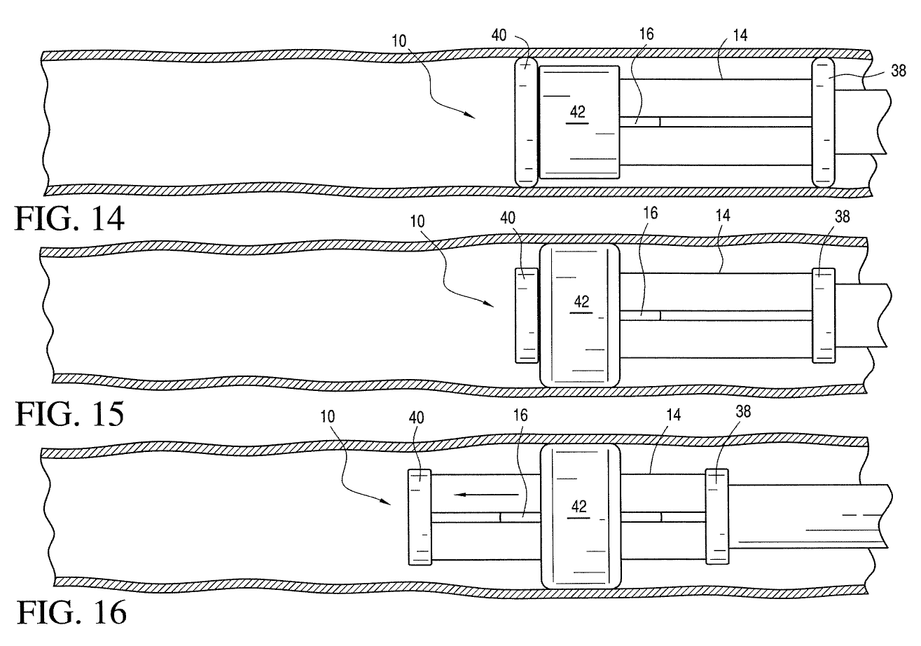

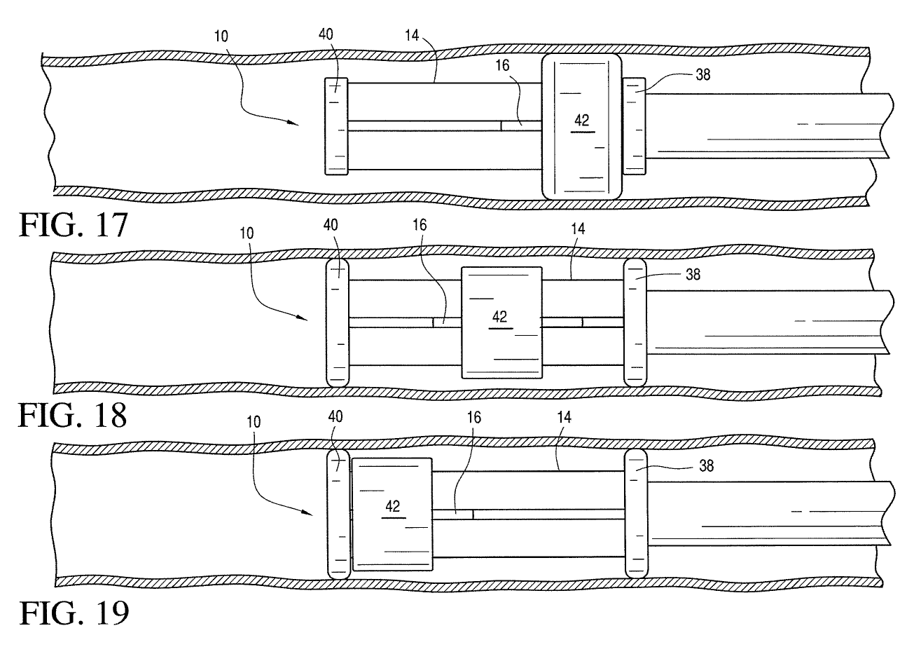

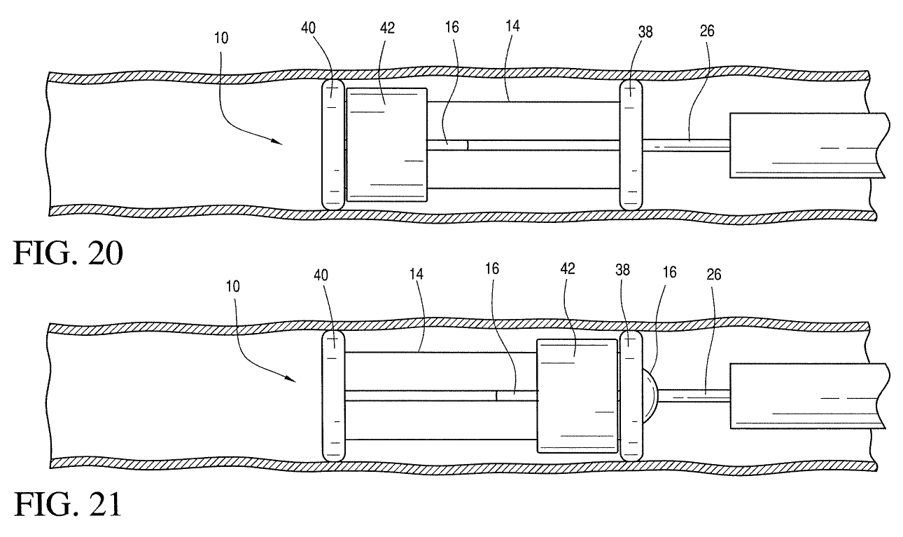

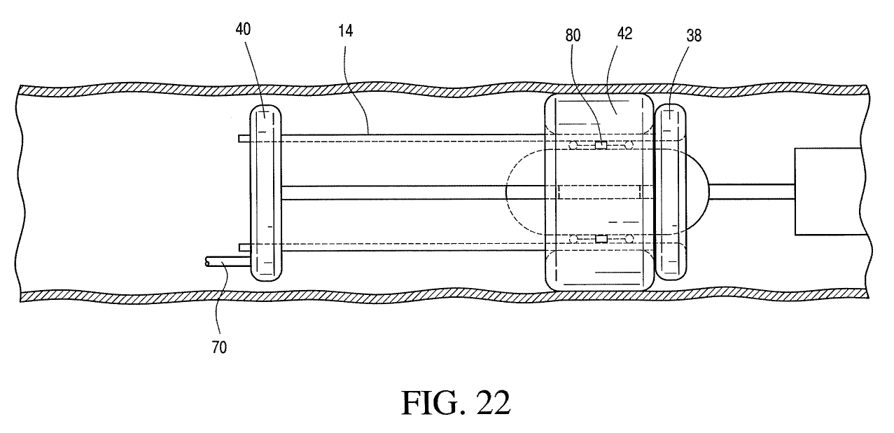

Referring now to FIGS. 8 and 9, the lumenal camera assembly 10 is designed to ride down the guide wire 76 and into position within the pylorus 24. The guide wire 76 is threaded through the lumenal camera assembly 10. The lumenal camera assembly 10 is then advanced into the patient's mouth and down the esophagus over the guide wire 76. As the lumenal camera assembly 10 is advanced blindly over the guide wire 76, the alternating balloon inflation process is preferably employed to get the lumenal camera assembly 10 to advance into the pylorus 24 (see FIGS. 11-21 which are described below in greater detail). Once the lumenal camera assembly 10 is in place, the insertion control tube 26 is removed by pulling on it while the first, second and third balloons 38, 40, 42 stay inflated which holds the lumenal camera assembly 10 in place. Alternately, a flexible endoscope may be used to advance the assembly down the guide wire that would eliminate the need to remove the insertion tube.

In accordance with an alternate embodiment, it might not be necessary to employ the guide wire as discussed above, and the lumenal camera assembly 10 may be guided to the pylorus 24 using the computer based control system 60 to inflate and deflate the balloon members 38, 40, 42. In the implementation of such a computer controlled device the user controls are simple controls like “advance”, “retract”, and “lock in place”.

More particularly, the control system 60 is employed to sequentially inflate and deflate the balloon members 38, 40, 42 of the lumenal camera assembly 10 as it is moved to a desired position within the pylorus 24. With the lumenal camera assembly 10 within the esophagus, and the first, second and third balloon members 38, 40, 42 inflated, the third balloon member 42 is deflated allowing the third balloon member 42 (and the pill cam 16) to move distally toward the second balloon member 40 by actuation of the linear motion device 80 (see FIGS. 12, 13 and 14). The first and second balloon members 38, 40 are then deflated while the third balloon member is inflated 42 (see FIG. 15). The housing 14 of the lumenal camera assembly 10 is then moved distally while the third balloon member 42 and the pill cam 16 remain in position due to the engagement of the third balloon member 42 while it is in its inflated state (see FIG. 16); thereby moving the first balloon member 38 to a position adjacent the third balloon member 42. The first and second balloon members 38, 40 are then inflated, while the third balloon member 42 is deflated. The third balloon member 42 (and the pill cam 16) is once again moved distally toward the second balloon member 40 (see FIG. 19). This process is repeated until the lumenal camera assembly 10 is properly positioned at which time the pill cam is moved to its exposed position adjacent the second end of the housing 14.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used system is obtained and if necessary cleaned. The system can then be sterilized. In one sterilization technique, the system is placed in a closed and sealed container, such as a plastic or TYVEK bag. The container and system are then placed in a field of radiation that can penetrate the container, such as gamma radiation, x-rays, or high-energy electrons. The radiation kills bacteria on the system and in the container. The sterilized system can then be stored in the sterile container. The sealed container keeps the system sterile until it is opened in the medical facility.

It is preferred that the device is sterilized. This can be done by any number of ways known to those skilled in the art including beta or gamma radiation, ethylene oxide or steam.

While the preferred embodiments have been shown and described, it will be understood that there is no intent to limit the invention by such disclosure, but rather, is intended to cover all modifications and alternate constructions falling within the spirit and scope of the invention.

1. A lumenal camera assembly, comprising:
a housing;
a pill cam supported by the housing;
wherein the housing is shaped and dimensioned for placement within a pylorus.

2. The lumenal camera assembly according to claim 1, further including a control tube secured to a back end of the lumenal camera assembly.

3. The lumenal camera assembly according to claim 1, wherein the housing includes a first end and a second end, the first end is provided with a first balloon member and the second end is similarly provided with a second balloon member.

4. The lumenal camera assembly according to claim 3, wherein the first balloon member and the second balloon member are substantially annular and are shaped and dimensioned for selective engagement with body members through which the lumenal camera assembly passes.

5. The lumenal camera assembly according to claim 3, further including a control system for inflation and deflation of the first balloon member and the second balloon member.

6. The lumenal camera assembly according to claim 3, wherein the housing includes a housing body which extends from the first balloon member to the second balloon member, and a third balloon member is mounted about the housing body for movement between the first balloon member and the second balloon member.

7. The lumenal camera assembly according to claim 6, further including a control system for inflation and deflation of the first balloon member, the second balloon member and the third balloon member.

8. The lumenal camera assembly according to claim 7, wherein the first balloon member, the second balloon member, and the third balloon member are substantially annular.

9. The lumenal camera assembly according to claim 7, wherein the pill cam is mounted within the housing for movement between the first end of the housing and the second end of the housing.

10. The lumenal camera assembly according to claim 9, wherein the pill cam is secured to the third balloon member such that the third balloon member is moved longitudinally along the housing with the pill cam.

11. The lumenal camera assembly according to claim 1, further including means for insufflation or suction.

12. A method for visualization of a gastric cavity, comprising:
transorally deploying a lumenal camera assembly composed of housing with a pill cam mounted thereto;
positioning the lumenal camera assembly within a pylorus such that the pill cam is capable of visualizing the gastric cavity.

13. The method according to claim 12, wherein the lumenal camera assembly includes a plurality of balloon members, and the step of positioning includes sequentially inflating and deflating the balloon members for positioning of the lumenal camera assembly within the pylorus.

14. The method according to claim 13, wherein the housing includes a first end and a second end, the first end is provided with a first balloon member and the second end is similarly provided with a second balloon member.

15. The method according to claim 14, wherein the first balloon member and the second balloon member are substantially annular and are shaped and dimensioned for selective engagement with body members through which the lumenal camera assembly passes.

16. The method according to claim 14, wherein the housing includes a housing body which extends from the first balloon member to the second balloon member, and a third balloon member is mounted about the housing body for movement between the first balloon member and the second balloon member.

17. The method according to claim 16, further including a control system for inflation and deflation of the first balloon member, the second balloon member and the third balloon member.

18. The method according to claim 17, wherein the pill cam is secured to the third balloon member such that the third balloon member is moved longitudinally along the housing with the pill cam.

19. The method according to claim 13, further including the step of positioning the lumenal camera assembly for obstruction of a small bowel from a stomach.

20. The method according to claim 13, further including the step of applying insufflation or suction via the lumenal camera assembly.
